(19) United States
(12) Patent Application Publication
(10) Pub. No.: US 2024/0376550 A1
(43) Pub. Date: Nov. 14, 2024

(54) TARGETED PHAGE THERAPY

(71) Applicant: The Regents of the University of California, Oakland, CA (US)

(72) Inventors: Bernd Schabl, San Diego, CA (US); Yi Duan, San Diego, CA (US); Sonja Lang, La Jolla, CA (US)

(21) Appl. No.: 17/783,263

(22) PCT Filed: Dec. 9, 2020
(86) PCT No.: PCT/US2020/064123

§ 371 Date: Jun. 7, 2022

Related U.S. Application Data
(60) Provisional application No. 62/946,182, filed on Dec. 10, 2019.

(51) Int. Cl. C12Q 1/689; C12Q 1/6874

(52) U.S. Cl. CPC C12Q 1/689; C12Q 1/6874; C12Q 2600/106; C12Q 2600/112

(57) ABSTRACT

Methods of diagnosing, monitoring progression or efficacy of therapy, and treating patients with hepatitis or other chronic liver diseases are provided.

### CROSS-REFERENCE TO RELATED APPLICATIONS

This application claims the benefit of the filing date of U.S. application No. 62/946,182, filed on Dec. 10, 2019, the disclosure of which is incorporated by reference herein.

### STATEMENT OF GOVERNMENT RIGHTS

This invention was made with government support under grant number AA026939 awarded by the National Institutes of Health. The government has certain rights in the invention.

### BACKGROUND

Alcohol-related liver disease is the most prevalent liver disease worldwide. Some patients develop hepatic inflammation, fibrosis, and liver cirrhosis, which increase risk for hepatocellular carcinoma and end-stage liver disease. Within this disease spectrum, alcoholic hepatitis is a severe acute-on-chronic liver failure, which is associated with 90-day mortality rates of 20%-50%; effective treatment options are lacking.

Intestinal dysbiosis and gut bacterial overgrowth contribute to development and progression of alcohol-related liver disease, but little is known about specific pathogens that are responsible for this process.

### SUMMARY

Extra-intestinal disease might be treated by targeting specific virulence factor-expressing bacteria in the gut. Bacterial virulence factors are proteins or peptides encoded by bacterial genes that help the organisms colonize the intestine or mediate disease; they have been predominantly evaluated in studies of epidemic and hypervirulent clinical strain isolates.

Intestinal bacterial metagenomes and genes that encode virulence factors in fecal samples from patients with alcoholic hepatitis were analyzed as described in more detail hereinbelow. The presence of virulence-related genes in commensal microbiota, and more specifically of 2 virulence factors encoded in the genome of Escherichia coli (ecpR (E. coli), kpsM (E. coli)), were independently associated with mortality in patients with alcoholic hepatitis, which is considered a metabolic, rather than an infectious, disease. Thus, certain virulence factors may distinguish between human health and disease, including distinguishing disease severity or mortality, and strategies to target virulence factor-positive bacteria may improve outcomes of patients, e.g., patients with alcohol-related liver disease. For example, bacteriophage that target E. coli expressing ecpR and/or kpsM genes may be employed as a therapeutic, for instance, formulated as a medicament for the treatment of people with hepatitis or alcohol-related liver disease, and administered in an amount of bacteriophage that target (and optionally lyse) E. coli expressing ecpR and/or kpsM genes.

In one embodiment, E. coli expressing ecpR and/or kpsM is a biomarker for disease severity/mortality.

In one embodiment, E. coli expressing ecpR and/or kpsM is a therapeutic target for agents including bacteriophage specific for E. coli expressing ecpR and/or kpsM.

In one embodiment, bacteriophage are selected that target E. coli expressing EcpR and/or KpsM and are manufactured as a medicament for the treatment of people with alcoholic hepatitis or other chronic liver diseases.

In one embodiment, a method of diagnosing a patient in need of treatment, e.g., a patient having hepatitis or other chronic liver disease, is provided that includes identifying the presence of E. coli expressing ecpR and/or kpsM in the patient.

In one embodiment, a patient in need of treatment, e.g., identified as having hepatitis or other chronic liver disease, or as having E. coli expressing ecpR and/or kpsM or an increased amount of E. coli expressing ecpR and/or kpsM, is administered a composition comprising an amount of bacteriophage selected as targeting E. coli expressing ecpR and/or kpsM.

Further provided is a medicament for the treatment of people with hepatitis or other chronic liver disease, comprising an amount of bacteriophage that target E. coli expressing ecpR and/or kpsM.

Also provided is a method of manufacturing a medicament for the treatment of people with hepatitis or other chronic liver disease, where bacteriophage are selected that target E. coli expressing ecpR and/or kpsM.

In one embodiment, a method of diagnosing a patient in need of treatment is provided by identifying the presence or amount of E. coli expressing ecpR and/or kpsM in a physiological sample. In one embodiment, the method includes identifying in a patient sample the presence or amount of E. coli expressing ecpR and/or kpsM, and in one embodiment providing treatment to the patient by formulating and delivering a medicament that includes an amount of bacteriophage selected to target E. coli expressing ecpR and/or kpsM.

Also provided is a method to prevent, inhibit or treat a mammal having hepatitis or other chronic liver disease, including administering a composition comprising an effective amount of bacteriophage selected to target E. coli expressing ecpR and/or kpsM.

In one embodiment, the amount of E. coli expressing ecpR and/or kpsM in a sample of a mammal is detected. In one embodiment, the sample is a fecal sample. In one embodiment, the sample is a non-fecal sample. In one embodiment, the mammal is a human. In one embodiment, the human has alcoholic liver disease. In one embodiment, the human has hepatitis or other chronic liver disease.

Further provided is a pharmaceutical composition comprising an amount of one or more bacteriophage selected to target E. coli expressing ecpR and/or kpsM.

A method to detect progression of liver disease in a mammal is provided. The method includes detecting over time the presence or amount of E. coli expressing ecpR and/or kpsM in a fecal, liver or blood sample of the mammal; and determining whether the presence or amount over time increases. In one embodiment, the mammal is a human. In one embodiment, the method further comprises administering a composition comprising bacteriophage that target E. coli expressing ecpR and/or kpsM. In one embodiment, the mammal is subjected to a liver transplant. In one embodiment, the mammal is administered an anti-inflammatory such as a corticosteroid. In one embodiment, the mammal is administered an hemorheologic agent. In one embodiment the method further comprises monitoring levels of E. coli expressing ecpR and/or kpsM after administration of the composition. In one embodiment, a cocktail of lytic phage is administered. In one embodiment, the composition is orally administered. In one embodiment, the composition is a tablet. In one embodiment, the composition is a sustained release dosage form.

In one embodiment, the phage that target E. coli expressing ecpR and/or kpsM target pili (fibriae). EcpR or other Ecp proteins, polysialic acid, KpsM, and/or the polysaccharide capsule. In one embodiment, the phage that target E. coli expressing ecpR and/or kpsM genes target FimA, G, F, and/or H.

### BRIEF DESCRIPTION OF FIGURES

FIGS. 1A-1D. Distribution of virulence-related genes in fecal samples from patients with alcohol-related liver disease. (A) Presence of 350 different virulence-related core genes in fecal samples from 81 patients with alcoholic hepatitis (AH), 41 patients with alcohol use disorder (AUD), and 9 controls (Ctrl). (B) Relative abundance of virulence factors found in fecal samples from 81 patients with alcoholic hepatitis, 41 patients with alcohol use disorder, and 9 non-alcoholic controls; factors are color-coded by genus of bacteria that produce them (C) Each bar presents data from one virulence factor-positive patient with alcoholic hepatitis (n=55). The x-axis reports the total number of virulence factor genes detected in fecal samples from each patient; patients are grouped by region (top of graph). The y-axis presents the relative abundance of virulence factors in each patient; factors are color-coded by genus of bacteria that produce them, as in panel B. (D) Percentage of patients with alcoholic hepatitis (n=81) positive for specific single virulence factors. The graph shows the top 60 most frequently detected virulence-related genes, color coded by genus of bacteria that expressed them. AH, alcoholic hepatitis, AUD, alcohol use disorder; Ctrl, non-alcoholic control P values in (A) were determined by Kruskal-Wallis test with Dunn's post-hoc test followed by false discovery rate (FDR) procedures.

FIGS. 2A-2G. Associations of virulence factors with mortality in patients with alcoholic hepatitis. (A) Kaplan-Meier curve of survival of patients with alcoholic hepatitis whose fecal samples were virulence factor (VF)-positive (n=55) or VF-negative (n=26). For the only patient who underwent liver transplantation, the transplantation date was considered as date of death. Patients were censored at the time point they were last seen alive. (B) Multivariate Cox regression adjusted for treatment with antibiotics and steroids. Only significant virulence factors are shown. Squares identify hazard ratios, with lower and upper 95% confidence intervals (CI) values shown as lines. Colors indicate the genus of bacteria that produces each specific virulence factor. (C, D) Core genome single-nucleotide polymorphism (SNP) tree of E. coli strains from patients with alcoholic hepatitis (n=81), showing phylogenetic diversity of kpsM and ecpR positive E. coli. (E) Principal component analysis (PCA), based on 573 different taxa on genus level. P=0.009, data from 81 patients with alcoholic hepatitis were included. The P value was determined by permutational multivariate analysis of variance (PERMANOVA). (F) Relative E. coli abundance in fecal samples from 81 patients with alcoholic hepatitis positive (n=26) or negative (n=55) for kpsM and/or ecpR. P value was determined by two-sided Mann-Whitney-Wilcoxon rank-sum test. (G) Multivariate Cox regression analysis of correlations between relative abundance of E. coli and mortality, adjusted for treatment with antibiotics, steroids, and the sequence batch (2 different runs). In the second row. E. coli abundance was additionally adjusted for the presence of the VFs kpsM and/or ecpR.

FIGS. 3A-3L. Virulence-related genes and associations with characteristics in patients with alcoholic hepatitis. (A) Association between virulence-related genes and time (days) between admission to the hospital and collection of fecal samples from 80 patients with alcoholic hepatitis; P−0.78. (B) Hospitalization time (days) for patients with alcoholic hepatitis either positive (n=55) or negative (n=26) for any virulence factor; P=48. (C) Associations of virulence-related genes with region of origin; P=0.95. (D) Virulence factor positivity in fecal samples from 81 patients with alcoholic hepatitis, by region of origin, P=0.52. (E) Proportions of patients with alcoholic hepatitis (n=68), with (n=13) or without (n=55) infection, whose fecal samples were positive (n=13) or negative (n=55) for kpsM; P=0.25. (F) Proportions of patients with alcoholic hepatitis (n=68), with (n=13) or without (n=55) infection, whose fecal samples were positive (n=19) or negative (n=49) for ecpR; P=0.49. (G) Number of virulence-related genes in fecal samples from patients with biopsy-proven alcoholic hepatitis, with (n=32) or without (n=13) liver cirrhosis; P=0.66. (H) Positivity for any virulence factor in fecal samples from patients with (n=32) or without (n=13) liver cirrhosis. P=0.49. (I) Correlation between the number of virulence-related genes and levels of zonulin in serum from 57 patients with alcoholic hepatitis; P=0.84. (J) Levels of zonulin in serum from virulence factor positive-patients (n=40) and virulence factor positive-patients (n=17), P=0.76. (K) Correlation between the number of virulence-related genes and levels of lipopolysaccharide binding protein (LPS-BP) in serum from 56 patients with alcoholic hepatitis, P=0.47. (L) Levels of LPS-BP in serum from virulence factor-positive patients (n=39) and virulence factor-negative patients (n=17); P=0.38. P values were determined by two-tailed Spearman's rank correlation coefficient (A, I, and K), Kruskal-Wallis test (C), two-sided Mann-Whitney-Wilcoxon rank-sum test (B, G, J, and L), and two-sided Fisher's exact test (D, E, F, and H).

FIGS. 4A-4D. Distribution of virulence-related genes in fecal samples from patients with alcohol-related liver disease. (A) Presence of 350 different virulence-related core genes in fecal samples from 81 patients with alcoholic hepatitis (AH), 41 patients with alcohol use disorder (AUD), and 9 controls (Ctrl). (B) Relative abundance of virulence factors found in fecal samples from 81 patients with alcoholic hepatitis, 41 patients with alcohol use disorder, and 9 non-alcoholic controls; factors are color-coded by genus of bacteria that produce them (C) Each bar presents data from one virulence factor-positive patient with alcoholic hepatitis (n=55). The x-axis reports the total number of virulence factor genes detected in fecal samples from each patient; patients are grouped by region (top of graph). The y-axis presents the relative abundance of virulence factors in each patient; factors are color-coded by genus of bacteria that produce them, as in panel B. (D) Percentage of patients with alcoholic hepatitis (n=81) positive for specific single virulence factors. The graph shows the top 60 most frequently detected virulence-related genes, color coded by genus of bacteria that expressed them. AH, alcoholic hepatitis, AUD, alcohol use disorder; Ctrl, non-alcoholic control. P values were determined by Kruskal-Wallis test with Dunn's post-hoc test followed by false discovery rate (FDR) procedures (A).

FIGS. 5A-5B. Correlations of virulence factors with mortality in patients with alcoholic hepatitis. (A) Kaplan-Meier curve of survival of patients with alcoholic hepatitis whose fecal samples were virulence factor (VF)—positive (n=55) or VF-negative (n=26). P=0.008. For the only patient who underwent liver transplantation, the transplantation date was considered as date of death. Patients were censored at the time point they were last seen alive. (B) Multivariate Cox regression adjusted for treatment with antibiotics and steroids. Only significant virulence factors are shown. Squares identify hazard ratios, with lower and upper 95% confidence intervals (CI) values shown as lines. Colors indicate the genus of bacteria that produces each specific virulence factor. P values were determined by two-sided Log-rank (Mantel-Cox) test (A) or two-side Wald test (B).

FIGS. 6A-7D. Associations of virulence factors and relative abundance of E. coli in alcoholic hepatitis patients. (A, B) Maximum likelihood phylogeny of E. coli strains (n=45, from 81 alcoholic hepatitis patients), showing phylogenetic distribution of ecpR and kpsM positive E. coli (n=25 and n=18, respectively) across the commensal (blue branches; Escherichia coli and Escherichia sp000208585) and pathogenic (black branches; Shigella flexneri and uropathogenic E. coli) lineages. (C) Principal component analysis (PCA), based on 1137 different taxa on genus level. P=0.001, data from 81 patients with alcoholic hepatitis were included. (D) Relative E. coli abundance in fecal samples from 81 patients with alcoholic hepatitis positive (n=26) or negative (n=55) for ecpR and/or kpsM P values were determined by permutational multivariate analysis of variance (PERMANOVA) (C) and two-sided Mann-Whitney-Wilcoxon rank-sum test (D).

FIGS. 7A-7F. Virulence-related genes and associations with hospitalization, patient origin and infection in patients with alcoholic hepatitis. (A) Association between virulence-related genes and time (days) between admission to the hospital and collection of fecal samples from 80 patients with alcoholic hepatitis; P=0.78. (B) Hospitalization time (days) for patients with alcoholic hepatitis either positive (n=55) or negative (n=26) for any virulence factor; P=48. (C) Associations of virulence-related genes with region of origin. P=0.95. (D) Virulence factor positivity in fecal samples from 81 patients with alcoholic hepatitis, by region of origin; P=0.52. (E) Proportions of patients with alcoholic hepatitis (n=68), with (n=13) or without (n=55) infection, whose fecal samples were positive (n=19) or negative (n=49) for ecpR; P=49. (F) Proportions of patients with alcoholic hepatitis (n=68), with (n=13) or without (n=55) infection, whose fecal samples were positive (n=13) or negative (n=55) for kpsM; P=0.25. P values were determined by two-tailed Spearman's rank correlation coefficient (A), two-sided Mann-Whitney-Wilcoxon rank-sum test (B), Kruskal-Wallis test (C), and two-sided Fisher's exact test (D. E and F).

FIGS. 8A-8F. Virulence-related genes and associations with liver cirrhosis and intestinal permeability in patients with alcoholic hepatitis. (A) Number of virulence-related genes in fecal samples from patients with biopsy-proven alcoholic hepatitis, with (n=32) or without (n=13) liver cirrhosis, P=0.66. (B) Positivity for any virulence factor in fecal samples from patients with (n=32) or without (n=13) liver cirrhosis. P=0.49. (C) Correlation between the number of virulence-related genes and levels of zonulin in serum from 57 patients with alcoholic hepatitis; P=0.84. (D) Levels of zonulin in serum from virulence factor positive-patients (n=40) and virulence factor negative-patients (n=17); P=0.76. (E) Correlation between the number of virulence-related genes and levels of lipopolysaccharide binding protein (LPS-BP) in serum from 56 patients with alcoholic hepatitis; P=0.47. (F) Levels of LPS-BP in serum from virulence factor-positive patients (n=39) and virulence factor-negative patients (n=17); P=0.38. P values were determined by two-sided Mann-Whitney-Wilcoxon rank-sum test (A, D, and F), two-sided Fisher's exact test (B) and two-tailed Spearman's rank correlation coefficient (C and E).

FIG. 9. Transplantation of feces from E. coli virulence factor-positive patients with alcoholic hepatitis exacerbates ethanol-induced liver disease in gnotobiotic mice. Serum level of ALT as marker of liver injury and disease. C57BL/6 germfree mice were colonized with feces from four different virulence factor-positive and four different virulence factor-negative patients with alcoholic hepatitis. After gnotobiotic mice were transplanted with the fecal samples, they were fed oral isocaloric (control) or chronic-binge ethanol diets.

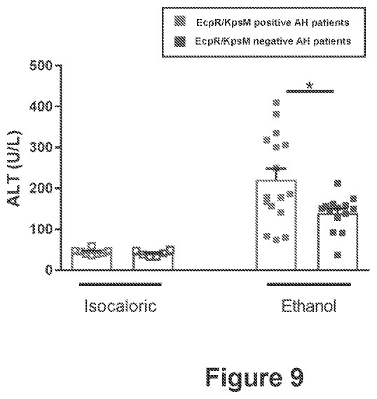

### DETAILED DESCRIPTION

Alcohol-related liver disease is the most prevalent liver disease worldwide. Some patients develop hepatic inflammation, fibrosis, and liver cirrhosis, which increases the risk for hepatocellular carcinoma and end-stage liver disease. Within this disease spectrum, alcoholic hepatitis is a severe acute-on-chronic liver failure, which is associated with 90-day mortality rates of 20%-50%; effective treatment options are lacking.

Intestinal dysbiosis and gut bacterial overgrowth contribute to development and progression of alcohol-related liver disease, but little is known about specific pathogens that are responsible for this process3. Cytolysin, a virulence factor expressed by Enterococcus faecalis, causes direct lysis of hepatocytes and liver damage, likely through its ability to form pores. Precision microbiota editing with bacteriophages reversed the liver damage induced by cytolysin-positive E. faecalis in mice with humanized microbiota and ethanol-induced liver disease. These findings indicated that extra-intestinal disease might be treated by targeting specific virulence factor-expressing bacteria in the gut. Bacterial virulence factors are proteins or peptides encoded by bacterial genes that help the organisms colonize the intestine or mediate disease; they have been predominantly evaluated in studies of epidemic and hypervirulent clinical strain isolates. Intestinal bacterial metagenomes and genes that encode virulence factors in fecal samples from patients with alcoholic hepatitis were analyzed.

### Definitions

As used herein, the term “isolated” in the context of phage refers to a phage which is separated from other molecules which are present in the natural source of the phage.

The term “purified” with respect to a bacteriophage means that the phage has been measurably increased in concentration by any purification process, including but not limited to, isolation from the environment or culture, e.g., isolation from culture following propagation and/or amplification, centrifugation, etc., thereby partially, substantially, nearly completely, or completely removing impurities, such as host cells and host cell components.

As used herein, the terms “therapeutic agent” and “therapeutic agents” refer to an agent, such as a bacteriophage or bacteriophage cocktail, that can be used in the treatment, management, or control of one or more symptoms of a disease or disorder.

As used herein, the terms “treat”. “treatment” and “treating” refer to obtaining a therapeutic benefit in a subject receiving a pharmaceutical composition. With respect to achieving a therapeutic benefit, the object is to eliminate, lessen, decrease the severity of, ameliorate, or slow the progression of the symptoms or underlying cause (e.g., bacterial infection) associated with the pathological condition or disorder. A “therapeutically effective amount” refers to that amount of a therapeutic agent, such as a phage cocktail pharmaceutical composition, sufficient to achieve at least one therapeutic benefit in a subject receiving the composition.

As used herein, the terms “prevent”, “prevention” and “preventing” refer to obtaining a prophylactic benefit in a subject receiving a pharmaceutical composition. With respect to achieving a prophylactic benefit, the object is to delay or prevent the symptoms or underlying cause (e.g., bacterial infection) associated with the pathological condition or disorder. A “prophylactically effective amount” refers to that amount of a prophylactic agent, such as a phage cocktail composition, sufficient to achieve at least one prophylactic benefit in a subject receiving the composition

Diagnostic. Predictive and Detection Methods

As described herein, the presence or amount of certain virulence factors, e.g., EcpR and/or KpsM, expressed in the gut microbiome, example, expressed by Enterobacteriaceae such as E. coli, in a physiological sample, such as a fecal sample, was found to be useful to predict whether a mammal with hepatitis, such as alcoholic hepatitis, has a short life expectancy, e.g., due to the severity of disease in the mammal, including alcoholic liver disease. The presence or amount of microorganisms having virulence genes and/or expressing certain virulence factors in a physiological sample, such as a fecal sample, may be useful to determine, for example, whether a mammal is in terminal phases of liver failure, whether liver disease is progressing, whether intervention is warranted or whether a therapy is effective.

The presence or amount of ecpR and or kpsM, or EcpR and/or KpsM, or organisms having those genes and/or expressing those genes, respectively, in a physiological sample may be detected by any means, direct or indirect. For example, the presence or amount of may be detected using an antibody to EcpR and/or KpsM. The presence or amount of EcpR and/or KpsM in a physiological sample may be detected using phage specific therefore. The presence or amount of organisms having ecpR and or kpsM in a physiological sample may be detected by detecting RNA that encodes EcpR and/or KpsM, or detecting genomic DNA that encodes EcpR and/or KpsM. In one embodiment, the method detects E. coli EcpR and/or KpsM in bacteria found in human intestines, but not the cytolysin of closely related bacterial species found in the gut. EcpR and/or KpsM may be detected by methods including but not limited to ELISA, Western blot or mass spectrometry.

In one embodiment, any probe or primer may be employed to detect ecpR and/or kpsM. In one embodiment, the primer may include KpsM_F: 5′-GCG CAT TTG CTG ATA CTG TTG-3′ (SEQ ID NO. 6); KpsM_R: 5′-CAT CCA GAC GAT AAG CAT GAG CA-3′ (SEQ ID NO: 7); EcpR_F: 5′-ATG GCA AAA TGA TTA CAG CAG-3′ (SEQ ID NO: 8); or EcpR_R: 5′-GTC CTT TAT AGA AGT AGG CGT C-3 (SEQ ID NO: 9). In one embodiment, one or more oligonucleotides are employed as primers in a nucleic acid amplification reaction. In one embodiment, at least one of the oligonucleotides has a length of about 5 to about 50, about 10 to about 25, about 15 to about 40, or about 15 to about 25 nucleotides. Thus, oligonucleotides within the scope of this disclosure include those having 1, 2, 3, 4, 5, or 6 nucleotide substitutions, lengths that are shorter, e.g., by 1, 2, 3, 4, 5, 6, 7, 8, 9 or 10 nucleotides, or lengths greater than, one of SEQ ID Nos. 6-9, or those having 1, 2, 3, 4, 5, or 6 nucleotide substitutions relative to 10, 15, 20 or 25 or ore consecutive nucleotides in one of SEQ ID Nos. 2 or 5, or the complement thereof, or a combination thereof.

Mammals having ecpR and/or kpsM or EcpR and/or KpsM detected in a physiological sample may be subjected to therapy, including phage therapy, to reduce the number of microorganisms expressing those factors.

### Phage Therapy

Bacteriophages, or phages for short, are viruses infecting bacteria that usually have a narrow host range. During infection of a bacterium, a phage has two principal life cycles it can enter—the lytic cycle and the lysogenic cycle. Both cycles are initiated by the attachment of the phage to a surface structure, which usually is species- and even strain-specific. After attachment, the phage injects its genetic material, which could be either DNA or RNA. After this injection, the phage can enter several different life cycles, with the lytic and lysogenic life cycle being the most common.

While all phages are capable of entering the lytic cycle (virulent phages), some phages (temperate phages) can also enter the lysogenic cycle. The lytic cycle results in production of phage particles; at the end of the cycle, the lysis cassette of the phage is expressed; this results in bacterial lysis and eventually in the release of new mature phage progeny.

Naturally occurring lytic phages are known for their antibacterial potential. To use bacteriophages as a therapeutic agent, naturally occurring bacteriophages that are able to lyse/kill E. coli expressing certain genes are isolated.

Phage therapy is safe and has been used in many clinical trials.

Exemplary phage useful in the therapeutic method include but are not limited to: those in any phage family that infects and optionally lyses or otherwise decreases the viability and/or replicative capacity of E. coli expressing ecpR and/or kpsM, or only removes the ecpR and/or kpsM containing virulence factors, e.g., including Myoviridae (non-enveloped, with head-tail (with a neck) geometries, and genomes are linear, double-stranded DNA, around 33-244 kb in length), Podoviridae (non-enveloped, with icosahedral and head-tail geometries where the double stranded DNA genome is linear, around 40-42 kb in length), Siphoviridae (non-enveloped, with icosahedral and head-tail geometries or a prolate capsid; genomes are double stranded and linear, around 50 kb in length). Exemplary E. coli targeting phage include but are not limited to PΦ9, e.g., VM75688, PΦ3, e.g., 350CIA, PΦ18, e.g., 258909, PΦ15, e.g., 278485-2, SΦ2, e.g., E1392-75, SΦ9, e.g., E7476, SΦ14, e.g., VM75688, EΦ2, e.g., 278485-2, EΦ4, e.g., VM75688, or EΦ9, e.g., E7476.

Other exemplary phage target pathogenicity islands in bacteria, e.g., large integrative elements that have one or more virulence genes that are absent in corresponding non-pathogenic bacteria of the same or closely related species. See, e.g., Desvaux et al., Front. Microbiol., 25 Sep. 2020, the disclosure of which is incorporated by reference herein.

### Exemplary Compositions and Therapeutic Methods

Thus, phage with the capacity of infecting bacteria and specifically, lytic phages, through infection of host bacteria, and are capable of decreasing populations of its host (target) bacteria without affecting other non-target bacterial strains, are useful in compositions and the methods described herein. By using more than one phage isolate or strain with a different specificity, resistance to the combination of phage is less likely. In one embodiment, phage are employed that bind to different receptors (e.g., using a phage cocktail), to lower the risk of developing resistance.

In the present disclosure, the choice of the lytic phage specific for E. coli decreases the population of target bacteria. Thus, phage can be employed to treat disorders associated with E. coli, such as liver disease. The phage is administered using, for instance, a delivery vehicle, such as a tablet, systemically or locally.

In one aspect, a composition comprising one phage isolate is employed. In one aspect, the composition comprises phage cocktails. In some embodiments, the composition comprises at least two different isolated strains of phage. In another aspect, the composition comprises a phage cocktail and a pharmaceutically acceptable carrier. In some embodiments, the pharmaceutical composition is formulated for systemic or local application. In some embodiments, the pharmaceutical composition comprises a sterile buffer, e.g., a buffer comprising about 0.05 M Tris-HCl, about 0.1M NaCl, and about 10 mM MgSO₄. In some embodiments, the composition further comprises an additional agent, e.g., an agent selected from the group consisting of an antibiotic agent, an anti-inflammatory agent, an antiviral agent, a local anesthetic agent, and a corticosteroid. In some embodiments, the composition is for use in treating a bacterial infection, and each of the phage strains is present in the composition in an amount corresponding to 10³ to 10²⁰ phage particles. In one embodiment, the dosage may be from 10³ to 10¹⁰ phage particles, 10⁵ to 10¹⁰ phage particles, 10¹⁰ to 10²⁰ phage particles, 10¹⁵ to 10²⁰ phage particles, or 10²⁰ to 10²⁵ phage particles. The daily dosage may be administered in one or more doses. In some embodiments, each of the phage strains is present in the composition in an amount corresponding to 10³ to 10⁵ phage, 10⁵ to 10⁸ phage 10⁵ to 10¹⁰ phage, or 10⁷ to 10⁹ phage particles and might be given once or multiple times daily. In some embodiments, the subject is a mammal, e.g., a human. In some embodiments, the treatment comprises administering a tablet or other orally compatible delivery vehicle having the composition.

In one aspect, cocktail compositions of different phage strains are administered to a human with liver disease, e.g., associated with alcohol use. The “cocktail” may comprise at least two different isolated strains of phage, for example, two, three, four, five, six, seven, eight, nine, ten, or more different isolated bacteriophage strains. The cocktail may be used alone or in further combination with other therapies, e.g., antibiotic agents and/or growth factors. In some embodiments, the phage cocktail comprises at least 2 phage strains, at least 3 phage strains, at least 4 phage strains, at least 5 phage strains, at least 6 phage strains, at least 7 phage strains, at least 8 phage stains, at least 9 phage strains, at least 10 phage strains, or more. In some embodiments, the phage cocktail comprises 2-20 phage strains, 2-15 phage strains, 2-10 phage strains, 3-8 phage strains, or 4-6 phage strains. In more embodiments, the combination does not impair or reduce (or does not substantially or significantly impair or reduce) infecting ability and/or lytic activity of the individual bacteriophage in the presence of distinct bacteriophage strains

The phage or phage cocktails are incorporated into a composition for the use in treatment of a disease. A cocktail of different phage strains, or a single phage isolate, may be combined with a pharmaceutically acceptable carrier, such as an excipient or stabilizer. e.g., to form a tablet. Examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin, hydrophilic polymers such as polyvinylpyrrolidone; amino acids such as glycine, glutamine, asparagine, arginine or lysine; monosaccharides, disaccharides, and other carbohydrates including glucose, mannose, or dextrins; sugar alcohols such as mannitol or sorbitol; salt-forming counterions such as sodium.

The bacteriophage or cocktail compositions may also be combined with one or more non-phage therapeutic and/or prophylactic agents, useful for the treatment and/or prevention of bacterial infections, as known in the art (e.g. one or more antibiotic agents). Other therapeutic and/or prophylactic agents that may be used in combination with the phage or phage cocktails include, but are not limited to, antibiotic agents, anti-inflammatory agents, antiviral agents, and corticosteroids. In some embodiments, the phage or phage cocktail is administered in the absence of a non-phage based antibiotic agent.

Standard antibiotics that may be used with pharmaceutical compositions comprising a phage cocktail include, but are not limited to, amikacin, gentamicin, kanamycin, neomycin, netilmicin, paromomycin, rhodostreptomycin, streptomycin, tobramycin, apramycin, rifamycin, naphthomycin, mupirocin, geldanamycin, ansamitocin, carbacephems, imipenem, meropenem, ertapenem, faropenem, doripenem, panipenem/betamipron, biapenem, PZ-601, cephalosporins, cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefonicid, cefprozil, cefuroxime, cefuzonam, cefmetazole, cefotetan, cefoxitin, cefcapene, cefdaloxime, cefdinir, cefditoren, cefetamet, cefixime, cefmenoxime, cefteram, ceftibuten, ceftiofur, ceftiolene, ceftizoxime, ceftriaxone, cefoperazone, ceftazidime latamoxef, cefclidine, cefepime, cefluprenam, cefoselis, cefozopran, cefpirome, cefquinome, flomoxef, ceftobiprole, azithromycin, clarithromycin, dirithromycin, erythromycin, roxithromycin, aztreonam, penicillin and penicillin derivatives, actinomycin, bacitracin, colistin, polymyxin B, cinoxacin, flumequine, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, garenoxacin, gemifloxacin, stifloxacin, trovalfloxacin, prulifloxacin, acetazolamide, benzolamide, bumetanide, celecoxib, chlorthalidone, clopamide, dichlorphenamide, dorzolamide, ethoxzolamide, furosemide, hydrochlorothiazide, indapamide, mafendide, mefruside, metolazone, probenecid, sulfacetamide, sulfadimethoxine, sulfadoxine, sulfanilamides, sulfamethoxazole, sulfasalazine, sultiame, sumatriptan, xipamide, tetracycline, chlortetracycline, oxytetracycline, doxycycline, lymecycline, meclocycline, methacycline, minocycline, rolitetracycline, methicillin, nafcillin, oxacilin, cloxacillin, vancomycin, teicoplanin, clindamycin, co-trimoxazole, flucloxacillin, dicloxacillin, ampicillin, amoxicillin and any combination thereof in amounts that are effective to additively or synergistically enhance the therapeutic effect of a composition having phage for a given infection.

In one embodiment, the compositions generally may include a sterile buffer, such as a sterile PBS, water, or saline buffer. One particular buffer comprises Tris-HCl, NaCl, and/or MgSO₄7H₂O, e.g., about 0.05 M Tris-HCl (pH 7.4-7.5), about 0.1 M NaCl, and/or about 10 mM MgSO₄7H₂O. In other embodiments, the formulation further comprises a buffer and 10 mM MgCl₂. In other embodiments, the phage containing formulation further comprises a buffer having about 5 mM to about 15 mM CaCl₂, e.g., about 10 mM CaCl₂).

In some embodiments, compositions are provided in a hermetically sealed container.

In one embodiment, the phage are formulated as an aqueous solution or gel. The composition may include water; esters, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and biphenyl alcohol; isoparaffins such as isooctane, isododecane and is hexadecane; silicone oils such as cyclomethicone, dimethicone, dimethicone cross-polymer, polysiloxanes and their derivatives, e.g., organomodified derivatives; polyols such as propylene glycol, glycerin, butylene glycol, pentylene glycol and hexylene glycol; or any combinations or mixtures of the foregoing. Aqueous vehicles may include one or more solvents miscible with water, including lower alcohols, such as ethanol, isopropanol, and the like.

### Formulations and Doses

The phage or cocktails thereof, can be formulated as pharmaceutical compositions and administered to a mammalian host, such as a human patient in a variety of forms adapted to the chosen route of administration, e.g., orally or parenterally, by intravenous, intramuscular, or subcutaneous routes. In one embodiment, the phage or cocktails thereof may be administered as a tablet. In one embodiment, the phage or cocktails thereof may be administered by infusion or injection. Solutions of the phage or cocktails thereof can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion may include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable tinder the conditions of manufacture and storage. The liquid carrier or vehicle can be a solvent or liquid dispersion medium comprising, for example, water, ethanol, a polyol (for example, glycerol, propylene gly col, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it may include isotonic agents, for example, sugars, buffers or sodium chloride. Prolonged absorption of the injectable compositions can be brought about by the use in the compositions of agents delaying absorption, for example, aluminum monostearate and gelatin.

The phage or cocktails thereof optionally in combination with another active compound may be administered parenterally, for example, intravenously, orally, intraperitoneally, intramuscularly, or subcutaneously. Such administration may be as a single bolus injection, multiple injections, or as a short- or long-duration infusion. Implantable devices (e.g., implantable infusion pumps) may also be employed for the periodic parenteral delivery over time of equivalent or varying dosages of the particular formulation. For such parenteral administration, the compounds (a conjugate or other active agent) may be formulated as a sterile solution in water or another suitable solvent or mixture of solvents. The solution may contain other substances such as salts, sugars (particularly glucose or mannitol), to make the solution isotonic with blood, buffering agents such as acetic, citric, and/or phosphoric acids and their sodium salts, and preservatives.

Thus, the phage or cocktails thereof or in combination with another active agent, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the phage or cocktails thereof optionally in combination with an active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of conjugate and optionally other active compound in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing an unit dosage form should be pharmaceutically acceptable and substantially non-toxic in the amounts employed. In addition, the phage or cocktails thereof optionally in combination with another active compound may be incorporated into sustained-release preparations and devices.

The phage or cocktails thereof optionally in combination with another active compound may also be administered intravenously or intraperitoneally by infusion or injection. Solutions of the phage or cocktails thereof optionally in combination with another active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle sue in the case of dispersions or by the use of surfactants. The prevention of the action of microorganisms during storage can be brought about by various antibacterial and antifungal agents, for example, parabens, chlorobutanol, phenol, sorbic acid, thimerosal, and the like. In many cases, it may be useful to include isotonic agents, for example, sugars, buffers or sodium chloride. Prolonged absorption of the injectable compositions can be brought about by the use in the compositions of agents delaying absorption, for example, aluminum monostearate and gelatin.

### EXEMPLARY EMBODIMENTS

The disclosure provides a medicament for the treatment of people with (alcoholic) hepatitis or other chronic liver disease, including an amount of bacteriophage that target Enterobacteriaceae, e.g., Escherichia such as E. coli, expressing ecpR and/or kpsM genes.

The disclosure also provides a method of manufacturing a medicament for the treatment of people with hepatitis or other chronic liver disease, where bacteriophage are selected to target Enterobacteriaceae, e.g., Escherichia such as E. coli, expressing ecpR and/or kpsM genes.

The disclosure further provides a method of diagnosing a patient in need of treatment, based on them having hepatitis or other chronic liver disease, and identifying the presence of Enterobacteriaceae, e.g., Escherichia such as E. coli, expressing ecpR and/or kpsM genes.

The disclosure provides a method of diagnosing a patient, based on them having hepatitis or other chronic liver disease, and identifying the presence of Enterobacteriaceae, e.g., Escherichia such as E. coli expressing ecpR and/or kpsM genes, and then providing treatment by formulating and delivering a medicament that includes an amount of bacteriophage selected to target Enterobacteriaceae, e.g., Escherichia such as E. coli expressing ecpR and/or kpsM genes.

The invention will be described by the following non-limiting examples.

### Example 1

Dysbiosis of the intestinal microbiota contributes to the progression of alcohol-related liver disease via unknown mechanisms. The presence of virulence-related genes in commensal microbiota, and more specifically of 2 virulence factors encoded in the genome of Escherichia coli, are independently associated with mortality in patients with alcoholic hepatitis, which is considered a metabolic, rather than an infectious, disease. Strategies to target virulence factor-positive bacteria might be developed to improve outcomes of patients with alcohol-related liver disease. Abundance of virulence factors might distinguish between human health and disease.

### Methods

### Study Population

Nine individuals without alcohol use disorder (controls), 41 patients with alcohol use disorder, and 81 patients with alcoholic hepatitis were included in the analysis. Patient cohorts have been described in detail (Brandl et al., 2018; Gao et al., 2019; Lang et al., 2019). Patients were identified as having alcohol use disorder if they fulfilled the DSM IV criteria (Ball et al., 1997). Controls were social drinkers who consumed less than 20 g alcohol per day. Neither controls nor patients with alcohol use disorder took antibiotics or immunosuppressive medication during the 2 months preceding enrollment. Inclusion and exclusion criteria of patients with alcoholic hepatitis are described in Duan et al (Nature, 575:505 (2019)), which is incorporated by reference herein). Patients with alcoholic hepatitis had clinical features of this disease. Liver biopsies were collected only if indicated as part of routine clinical care for the purpose of alcoholic hepatitis diagnosis. Histologic features of biopsies supported diagnoses of alcoholic hepatitis. The protocol was approved by the Ethics Committee of each participating center. Written informed consent was obtained from each subject.

### Shotgun Metagenomics Analysis

DNA was extracted from human stool samples using FastDNA Spin Kit for Soil (MP-Biomedicals). Whole-genome shotgun metagenomic sequencing was performed on Illumina HiSeq 4000 generating 150 bp pair-end reads. Quality control of shotgun metagenomics reads was performed using KneadData version 0.7.2. Metagenomic Phylogenetic Analysis 2 (MetaPhlAn2) version 2.7.7 was used to profile the composition of the microbial communities (Truong et al., 2015). The HMP Unified Metabolic Analysis Network 2 (HUMAnN2) version 0.11.1 was used to profile microbial pathways (Franzosa et al., 2018). The MetaCyc database was used for microbial pathway analysis (Caspi et al., 2018). Each HUMAnN2 abundance output was normalized for relative abundance (the counts for each sample sum to 100).

### Analysis of Virulence-Related Genes

### Data Availability

Sequence data were deposited in the European Nucleotide Archive tinder accession numbers ERP106878.

### Statistical Analysis

Results are expressed as median and range unless stated otherwise. Two groups were compared using the Mann-Whitney-Wilcoxon rank-sum test for continuous and Fisher's exact test for categorical variables. Three or more groups were compared using the Kruskal-Wallis test with Dunn's post-hoc test for continuous and Fisher's exact test for categorical variables, each followed by false discovery rate (FDR) procedures to correct for multiple comparisons. We used univariate and multivariate Cox regression analysis to assess associations of virulence factors with 180-day mortality. The multivariate model was adjusted for treatment with antibiotics and steroids. Kaplan-Meier curves were used to compare survival between virulence factor-positive and virulence factor-negative patients with alcoholic hepatitis. For the only patient who underwent liver transplantation, the transplantation date was considered as the date of death. Patients were censored at the time point they were last seen alive. Spearman's rank correlation coefficient was used to investigate associations between 2 continuous variables. To investigate gut microbiota profiles among patients with alcoholic hepatitis that were either positive or negative for specific virulence factors, we calculated the logarithmic relative abundance of all intestinal bacteria on the genus level. Euclidian distance was determined for the principal component analysis (PCA) and P values were determined by permutational multivariate analysis of variance (PERMANOVA). Statistical analyses were performed using R statistical software, R version 3.5.1, 2018 the R Foundation for Statistical Computing. A P<0.05 was considered to be statistically significant (adjusted for multiple comparison when performing multiple tests). All statistical tests were 2-sided.

### Introduction and Results

Alcohol-related liver disease is the most prevalent liver disease worldwide. Some patients develop hepatic inflammation, fibrosis, and liver cirrhosis, which increase risk for hepatocellular carcinoma and end-stage liver disease (Seitz et al., 2018). Within this disease spectrum, alcoholic hepatitis is a severe acute-on-chronic liver failure, which is associated with 90-day mortality rates of 20%-50%; effective treatment options are lacking (Lucey et al., 2009).

Intestinal dysbiosis and gut bacterial overgrowth contribute to development and progression of alcohol-related liver disease, but little is known about specific pathogens that are responsible for this process (Sarin et al., 2019). Recently it was found that cytolysin, a virulence factor expressed by Enterococcus faecalis, causes direct lysis of hepatocytes and liver damage, likely through its ability to form pores. Precision microbiota editing with bacteriophages reversed the liver damage induced by cytolysin-positive E. faecalis in mice with humanized microbiota and ethanol-induced liver disease. These findings indicated that extra-intestinal disease might be treated by targeting specific virulence factor-expressing bacteria in the gut. Bacterial virulence factors are proteins or peptides encoded by bacterial genes that help the organisms colonize the intestine or mediate disease; they have been predominantly evaluated in studies of epidemic and hypervirulent clinical strain isolates. We analyzed intestinal bacterial metagenomes and genes that encode virulence factors in fecal samples from patients with alcoholic hepatitis.

Shotgun sequencing of fecal samples from 81 patients with alcoholic hepatitis, 41 patients with alcohol use disorder, and 9 individuals who were not alcoholics (controls) (Table 1) was performed; and sequences aligned with those in the virulence factor gene database (VFDB) (Chen et al., 2016). These patients with alcoholic hepatitis from 7 regions in Europe, the United States, and Mexico were enrolled in a multi-center observational study at 12 centers. The median hospitalization time before collection of fecal samples was 4 days (range, 0-24 days) (Table 2). The median model for end-stage liver disease (MELD) score of the patients with alcoholic hepatitis was 24; most patients with available liver biopsies had liver cirrhosis (Table 2).

TABLE 1
Demographic and laboratory parameters of the study cohort Values are
presented as median with range in brackets for continuous variables

| | Controls | | Alcohol use disorder | | | |
|---|---|---|---|---|---|---|
| Variables | (0 = 9) | | (n = 41) | | Alcoholic hepatitis | |
| Sex (% male), n (%), | 7 | (77.8) | 34 | (82.9) | 54 | (67.5) |
| n = 130 | | | | | | |
| Age (years), n = 130 | 51.0 | (27.0-71.0) | 44.0 | (27.0-67.0) | 51.3 | (30.0-75.0) |
| BMI (kg/m²), n = 118 | 22.9 | (18.8-29.2) | 24.0 | (17,9-37.0) | 27.5 | (19.0-48.3) |
| Creatinine (mg/dL), | | | 0.8 | (0.5-1.3) | 0.8 | (0.3-8.1) |
| n = 118 | | | | | | |
| Bilirubin (mg/dL), | | | 0.5 | (0.2-1.5) | 16.3 | (2.5-38.6) |
| n = 118 | | | | | | |
| AST (IU/L), n = 121 | | | 36.0 | (15.0-283.0) | 132.0 | (41.0-406.0) |
| ALT (IU/L), n = 121 | | | 37.0 | (9.0-184.0) | 44.5 | (15.0-216.0) |
| Albumin (g/dL), | | | 4.5 | (2.2-5.2) | 2.4 | (1.3-4.1) |
| n = 112 | | | | | | |
| INR, n = 116 | | | 0.9 | (0.8-1.3) | 1.8 | (1.0-3.7) |
| GGT (IU/L), n = 77 | | | 43.0 | (4.0-1131.0) | 188.0 | (33.0-3632.0) |
| Platelet count (10⁹/L), | | | 222.0 | (21.0-434.0) | 122.5 | (21.0-447.0) |
| n = 122 | | | | | | |
| FIB-4, n = 115 | | | 1.3 | (0.4-21.4) | 8.0 | (1.4-66.3) |
| FIB-4 > 3.25 (F3-F4), | | | 6 | (16.2) | 71 | (92.2) |
| n (%) | | | | | | |

or number and percentage in brackets for categorical variables. Percentages are calculated based on the actual number of patients in each group where the respective data was available. The number of subjects for which the respective data was available is indicated in the first column. BMI, body, mass index; AST, aspartate aminotransferase; ALT, alanine aminotransferase; INR, international normalized ratio; GGT, gamma-glutamyl transferase; MELD, model for end-stage liver disease; FIB-4, fibrosis-4 index

TABLE 2
Characteristics of patients with alcoholic hepatitis (n = 81)
Treatment at admission

| | | |
|---|---|---|
| Steroids, n (%), n = 79 | 31 | (39.2) |
| Pentoxifylline, n (%), n = 65 | 6 | (9.2) |
| Antibiotics, n (%), n = 79 | 40 | (50.6) |
| Proton pump inhibitors, n (%), n = 42 | 5 | (11.9) |

Clinical characteristics

| Hospitalization time prior sampling (d) | 4 | (0-24) |
|---|---|---|
| Infections, n (%), n = 68 | 13 | (19.1) |
| Blood stream infection | 7 | |
| Pneumonia | 4 | |
| Urinary tract | 3 | |
| SBP | 2 | |
| Unknown | 1 | |
| Abstinent at day 30, n = 47 | | |
| Abstinent, n (%) | 40 | (85.1) |
| Reduced alcohol consumption, n (%) | 4 | (8.5) |
| Continued alcohol abuse, n (%) | 3 | (6.4) |

| | |
|---|---|
| Stage of Fibrosis, n (%), n = 45 0/1/2/3/4 | 2 (4.4)/0 (0.0)/5 (11.1)/ |
| | 6 (13.3)/32 (71.1) |

Clinical scores and outcome

| MELD, median (range), n = 79 | 23.7 | (11.7-45.9) |
|---|---|---|
| MELD > 21, n (%) | 62 | (78.5) |
| Child-Pugh stage, n (%), n = 78 A/B/C | 1 (1.3)/24 (30.8)/53 (67.9) | |
| Maddrey's DE, median (range), n = 65 | 59.3 | (−3.8-239.0) |
| Maddrey's DF > 32, n (%) | 57 | (87.7) |
| ABIC, median (range), n = 82 | 8.3 | (5.5-11.0) |
| 30 day mortality rate, n (%), n = 73 | 8 | (11.0) |
| 90 day mortality rate, n (%), n = 53 | 13 | (24.5) |

Causes of death within 180 days

| | |
|---|---|
| Liver failure* | 10 |
| Gastrointestinal bleeding | 3 |
| Septic shock | 2 |
| Unknown | 3 |

*One patient underwent liver transplantation.
Notes:
antibiotics include prophylactic antibiotics. Values are presented as median (range) for continuous variables or number (percentage) for categorical variables.
Percentages are calculated based on the actual number of patients in each group where the respective data was available. Individual persons can have more then one reason for infections. The number of subjects for which the respective data was available is indicated in the first column.

MELD, model for end-stage liver disease; DF, discriminant function; ABIC, Age, serum bilirubin, INR, and serum creatinine score; SPB, spontaneous bacterial peritonitis.

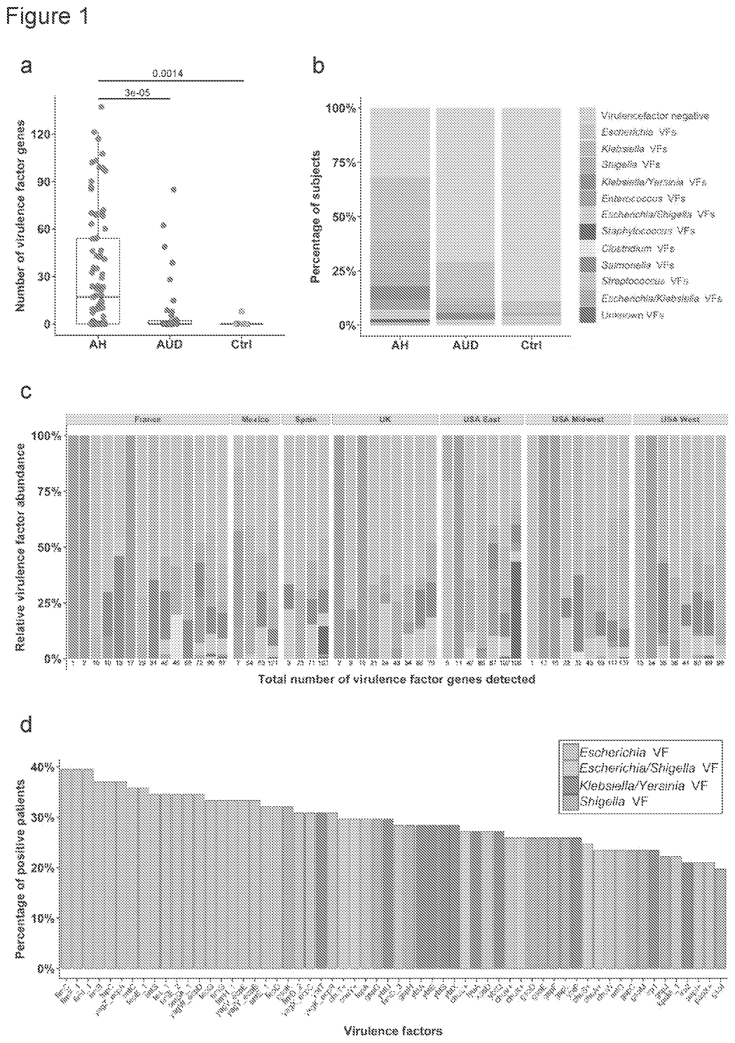

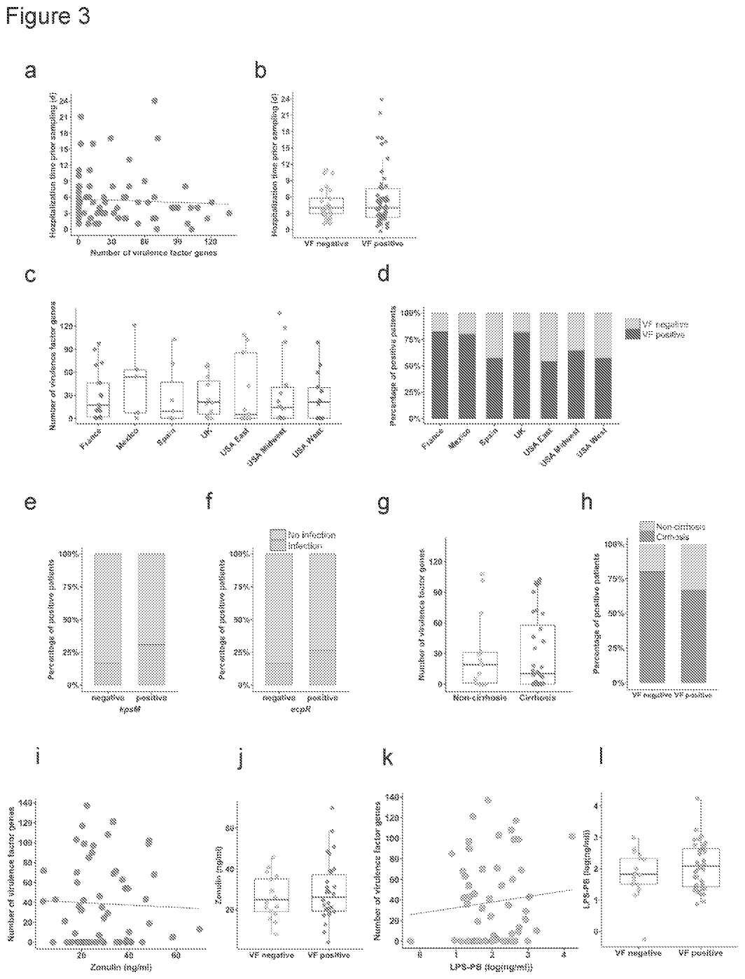

The core virulence factor genes included 350 different, experimentally verified genes that encode virulence factors. Significantly more virulence factors were found in fecal metagenomes from patients with alcoholic hepatitis than patients with alcohol use disorder or controls (FIG. 1A). The median hospitalization time of patients with alcoholic hepatitis was not associated with increased copies of genes encoding virulence factors, indicating that they were not acquired during hospitalization (FIGS. 3A, 3B). Overall, fecal samples from 68% of patients with alcoholic hepatitis had at least one virulence factor gene, compared with 29% of patients with alcohol use disorder and 11% of controls (FIG. 1B). There was heterogeneity in virulence factor genes in samples from each patient with alcoholic hepatitis, fecal samples from most patients with any one virulence factor gene were found to have other virulence factor genes, independent from the center where the patient was enrolled (FIG. 1C, FIGS. 3C,3D). The most frequently observed virulence factors were derived from Escherichia, followed by Klebsiella, Shigella, Klebsiella or Yersinia, and then Enterococcus (FIGS. 1B, 1D).

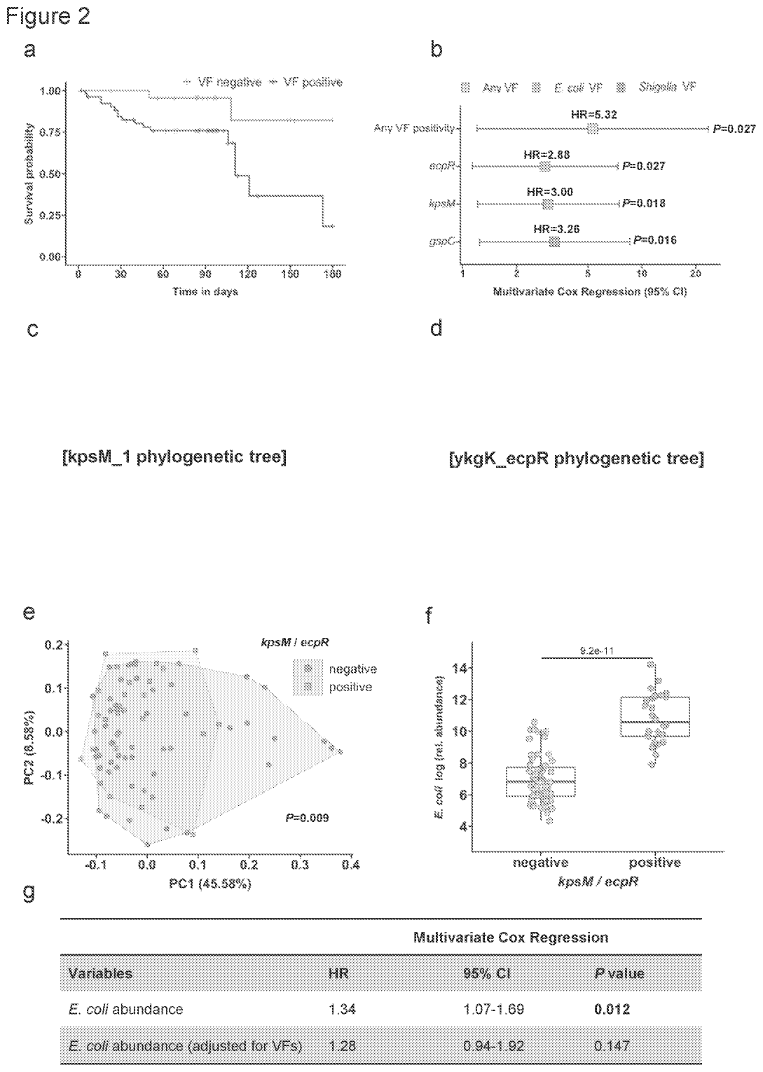

In patients with alcoholic hepatitis, a fecal sample with any virulence factor gene was associated with decreased probability of surviving 180 days (FIG. 2A); 82% of virulence factor-negative patients were alive after 180 days following enrollment, compared with 18% of virulence factor-positive patients. In the univariate Cox regression analysis, a fecal sample with any virulence factor gene, with any Escherichia virulence factor gene, or with 1 of 6 specific virulence factor genes was associated with 180-day mortality (Table 3). After adjusting for antibiotic and steroid treatment in the multivariate Cox regression analysis, fecal samples with any virulence factor gene remained associated with increased risk of death, with an adjusted hazard ratio of 5.3 (95% CI 1.2-23.5, P=0.027; FIG. 2B). Specifically, among all 350 core virulence factor genes, fecal samples containing ecpR (E. coli), kpsM (E. coli), or gspC (Shigella dysenteriae) were independently associated with mortality (FIG. 2b).

TABLE 3
Univariate Cox regression analysis of virulence factors associated
with 180-day mortality in patients with alcoholic hepatitis

| Univariate Cox | | | | |
|---|---|---|---|---|
| regression | Organism | HR | 95% CI | P value |
| Any virulence factor | | 5.9 | 1.3-25.7 | 0.019 |
| positivity | | | | |
| Escherichia virulence | | 2.9 | 1.0-8.1 | 0.041 |
| factor positivity | | | | |
| ecpR | E. coli | 3.0 | 1.2-7.5 | 0.019 |
| ecpD | E. coli | 2.7 | 1.1-6.8 | 0.031 |
| kpsM | E. coli | 3.0 | 1.2-7.5 | 0.017 |
| yhtX | K. pneumoniae/ | 2.7 | 1.1-6.7 | 0.033 |
| | Y. pestis | | | |
| gspC | S. dysenterige | 3.5 | 1.4-9.0 | 0.010 |
| chuS | E. coli/ | 2.7 | 1.1-6.7 | 0.034 |
| | S. dysenteriae | | | |

Significance is indicated in bold.
Patients were censored at the time point they were last seen alive. P values were determined by two-sided Wald test.
HR, hazard ratio;
CI, confidence interval

The core genome single-nucleotide polymorphism (SNP) tree of ecpR- and kpsM-positive E. coli had broad phylogenetic diversity (FIGS. 2C, 2D); these virulence factor genes were not associated with infection immediately before or during hospitalization (FIGS. 3E, 3F; Table 2). Patients positive for ecpR and/or kpsM (32% of patients with alcoholic hepatitis) had a microbiome that differed significantly from that of non-carriers, when we analyzed the relative abundance of fecal bacterial populations in the metagenomic dataset (FIG. 2E). In particular, the presence of genes encoding virulence factors from E. coli highly correlated with the relative abundance of E. coli (FIG. 2F). Higher E. coli abundance itself was independently associated with 180-day mortality. However, when we adjusted for detection of ecpR and/or kpsM, this association was no longer statistically significant (FIG. 2G). This indicates that the abundance and expression of genes encoding E. coli virulence factors each affect risk of death in these patients.

The presence of liver cirrhosis was not associated with higher copy numbers of virulence factor genes (FIGS. 3g, 3H). Furthermore, levels of zonulin (a marker for gut barrier dysfunction) and lipopolysaccharide binding protein (LPS-BP) (a marker of the systemic immune response to LPS) in serum samples were not associated with the number or presence of any virulence factor genes (FIGS. 3I-3L). Virulence factors therefore do not seem to increase intestinal permeability or translocation of LPS.

This study may be the first to investigate the correlation between clinical outcomes and specific virulence-related genes in the gut metagenomes from patients with a metabolic and non-infectious disease. Among patients with liver cirrhosis, E. coli infections and in particular spontaneous bacterial peritonitis are frequent complications with high mortality. In this studs, the presence of ecpR and kpsM in the gut microbiota was not associated with increased rates of infection. The broad phylogenetic diversity indicates that ecpR and kpsM presence is a variable trait among E. coli isolates, and ecpR- or kpsM-positive E. coli are diverse and largely non-epidemic commensal strains. The low frequency of ecpR and kpsM in fecal samples (22% and 31%, respectively) indicates the non-epidemic or hypervirulent nature of ecpR- and kpsM-positive E. coli strains, since epidemic isolates have typically higher frequencies of virulence genes (Bert et al., 2010).

Little is known about the specific environmental conditions leading to enrichment of virulence-related genes. Profound dysregulation of bile acid metabolism, which we observed in patients with alcoholic hepatitis, could be one possible explanation for this expansion. The syndrome of alcoholic hepatitis develops rapidly and although most patients have underlying chronic liver disease, they are not repeatedly hospitalized. Liver failure was the main cause of death among the 18 patients who died within 180 days, followed by gastrointestinal bleeding; only 2 patients died from sepsis (Table 2). Therefore, mechanisms beyond infection contribute to the association between mortality and the presence of these virulence factors in patients with alcoholic hepatitis.

Metagenome sequencing might not fully capture the presence of virulence factors among all bacterial populations in the gut; increased cytolysin in fecal samples from patients with alcoholic hepatitis, which was detected by quantitative PCR in Duan et al. (2019) was not observed by shotgun sequencing. Therefore, a combination of different techniques might be necessary to detect genes that encode virulence factors in bacteria of low abundance. The strength of this study is that it included patients with alcoholic hepatitis participating in a multi-center observational trial, which reduced the chances that the virulence-related genes studied are region specific.

Most studies investigating associations of the gut microbiota and disease have focused on diversity, relative abundance and metabolic profiles of specific microorganisms. Assessing the abundance of specific virulence factors adds an additional layer of complexity that might lead to new diagnostic biomarkers and treatments for diseases that are not considered classic infections. Abundance of virulence factors might distinguish between human health and disease.

### Example 2

Alcohol-related liver disease is the most prevalent liver disease worldwide and the main cause of liver-related mortality (Lozano et al., 2012; Rehm et al. 2014; Rehm et al., 2013). Alcohol-related liver disease has recently become the leading cause of liver transplantation in the United States (Lee et al., 2018). Some patients develop hepatic inflammation, fibrosis, and liver cirrhosis with increased risk for hepatocellular carcinoma (Seitz et al., 2018; Seki et al., 2015). Within this disease spectrum, alcoholic hepatitis is a severe acute-on-chronic liver failure, which is associated with 90-day mortality rates of 20%-50% (Maddrey et al., 2003; Mathurin et al., 2003; Dominguez et al., 2008; Thursz et al., 2015; Forrest et al., 2005). Early liver transplantation is the only curative therapy, but is solely available at select centers, to a limited group of patients (Thursz et al., 2015; Mathurin et al., 2012; Lucey et al., 2009). Therefore, prognostic biomarkers and new therapeutic targets are urgently needed for this severe and deadly disease.

Chronic alcohol consumption results in intestinal microbial dysbiosis (Chen et al., 2015; Stärkel et al., 2016) which contributes to the development and progression of alcohol-related liver disease (Uesugi et al., 2001; Puri et al., 2018; Zhong et al., 2019). Transplantation of intestinal microbiota from alcoholic hepatitis patients promotes ethanol-induced liver disease in mice (Llopis et al., 2(16). A pilot study showed that the survival rate in steroid-ineligible patients with alcoholic hepatitis was improved by fecal microbiota transplantation (Philips et al., 2017). These findings indicate that alcohol-associated liver disease can be transmitted via fecal microbiota.

Most studies investigating associations of the gut microbiota and diseases have focused on diversity, relative abundance and metabolic profiles of specific microorganisms (Chen et al., 2011; Caussy et al., 2018; Ponziani et al., 2019). However, little is known about the specific pathogens, especially the exact contributing factors of the pathogens that are responsible for the disease development and progression. Bacterial virulence factors are proteins or peptides encoded by bacterial genes that help the organisms colonize the intestine or mediate disease (Hochhut et al., 2005; Lee et al., 2001; Burrack et al., 2007). They have been predominantly evaluated in studies of epidemic and hypervirulent clinical strain isolates (Kim et al., 1992; Cirioni et al., 2007; Blackburn et al., 2009), but not in metabolic, non-infectious diseases.

Recently it was found that cytolysin, a virulence factor expressed by Enterococcus faecalis (E. faecalis), causes direct lysis of hepatocytes and liver damage, likely through its ability to form pores. Precision microbiota editing with bacteriophages reversed the liver damage induced by cytolysin-positive E. faecalis in mice with humanized microbiota and ethanol-induced liver disease (Duan et al., 2019). These findings indicated that extra-intestinal diseases might be treated by targeting specific virulence factor-expressing bacteria in the gut.

In the present study, intestinal bacterial metagenomes and genes that encode virulence factors in fecal samples were analyzed, from a well described, multi-center cohort of patients with alcoholic hepatitis. The associations between virulence-related genes and disease outcomes in those patients were investigated.

### Experimental Procedures

### Human Subjects

Nine individuals without alcohol use disorder (controls), 41 patients with alcohol use disorder, and 81 patients with alcoholic hepatitis were included in the analysis. Patient cohorts have been described in Chu et al., 2019; Lang et al., 2019; and Gao et al., 2019). Patients were identified as having alcohol use disorder if they fulfilled the DSM IV criteria (Ball et al., 1997). Controls were social drinkers who consumed less than 20 g alcohol per day. Neither controls nor patients with alcohol use disorder took antibiotics or immunosuppressive medication during the 2 months preceding enrollment. Inclusion and exclusion criteria of patients with alcoholic hepatitis are described in Duan et al. (2019). Patients with alcoholic hepatitis had clinical features of this disease. Liver biopsies were collected only if indicated as part of routine clinical care for the purpose of alcoholic hepatitis diagnosis. Histologic features of biopsies supported diagnoses of alcoholic hepatitis. The protocol was approved by the Ethics Committee of each participating center. Written informed consent was obtained from each subject.

### Shotgun Metagenomics Analysis

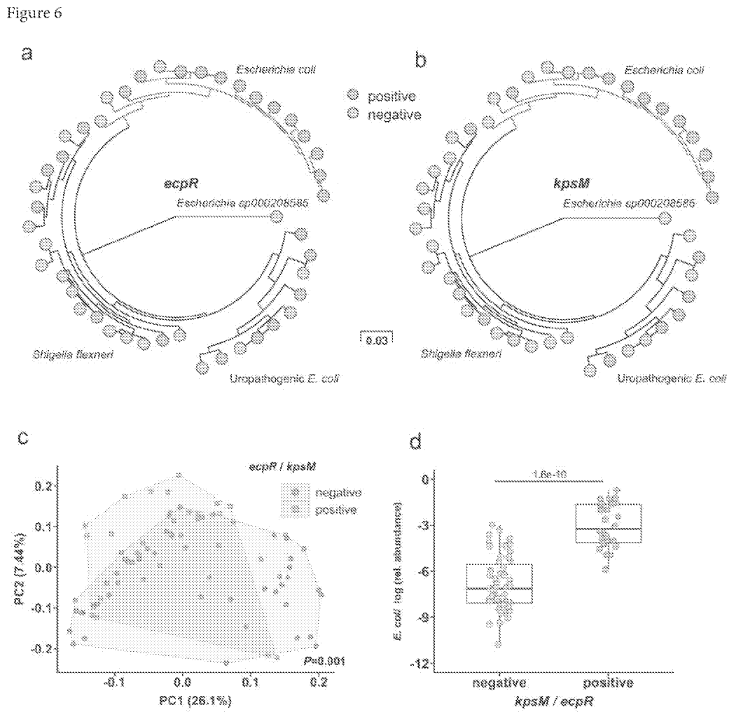

DNA was extracted from human stool samples using FastDNA Spin Kit for Soil (MP-Biomedicals). Whole-genome shotgun metagenomic sequencing was performed on Illumina HiSeq 4000 generating 150 bp pair-end reads. Quality control of shotgun metagenomics reads was performed (Shao et al., 2019). Taxonomic classification of the processed metagenomics reads was performed using Kraken (Wood et al., 2014) against genomes of the Human Gastrointestinal Bacteria Genome Collection (Forrester et al., 2019). The maximum-likelihood phylogenetic trees of Escherichia coli shown in FIGS. 6A and 6B were built by applying RAxML (Stamatakis et al., 2014) (internally in StrainPhlAn (Truong et al., 2017) on the alignment of E. coli marker genes, using default parameters and with the options ‘-alignment_program mafft’ and ‘-relaxed_parameters3’. The major lineages were taxonomically assigned to GTDB Parks et al., 2018) species clusters using GTDB r89 representative genomes.

### Analysis of Virulence-Related Genes

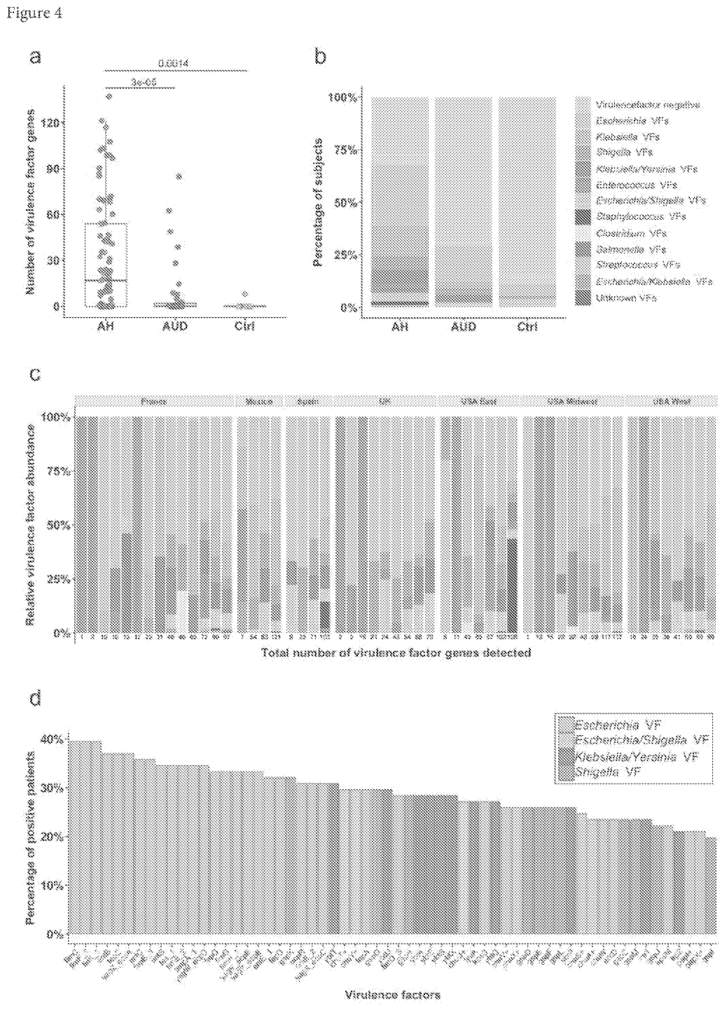

Quality-filtered metagenomic reads were screened for the presence of genes encoding for bacterial virulence factors using ARIBA (Hunt et al., 2017) with “-min_scaff_depth 1”, and otherwise default parameters. As previously described in Shao et al., VFDB core virulence genes were grouped into clusters based on 90% nucleotide identity, and then included to build a BLAST database for virulence-factor screening. The detected virulence genes were grouped by their host genera as found in VFDB. All potential host genera were shown (e.g., Klebsiella/Yersinia in FIGS. 4B and 4D) if the virulence factor gene cluster was found in multiple genera.

### Data Availability

Sequence data were deposited in the European Nucleotide Archive under accession numbers ERP106878.

### Statistical Analysis

Results are expressed as median and range unless stated otherwise. Two groups were compared using the Mann-Whitney-Wilcoxon rank-sum test for continuous and Fisher's exact test for categorical variables. Three or more groups were compared using the Kruskal-Wallis test with Dunn's post-hoc test for continuous and Fisher's exact test for categorical variables, each followed by false discovery rate (FDR) procedures to correct for multiple comparisons. Univariate and multivariate Cox regression analyses were used to assess associations of virulence factors with 180-day mortality. The multivariate model was adjusted for treatment with antibiotics and steroids. Kaplan-Meier curves were used to compare survival between virulence factor-positive and virulence factor-negative patients with alcoholic hepatitis. For the only patient who underwent liver transplantation, the transplantation date was considered as the date of death. Patients were censored at the time point they were last seen alive Spearman's rank correlation coefficient was used to investigate associations between two continuous variables. To investigate gut microbiota profiles among patients with alcoholic hepatitis that were either positive or negative for specific virulence factors, the logarithmic relative abundance of all intestinal bacteria on the genus level was calculated. Jaccard dissimilarity matrices were determined for the principal component analysis (PCA) and P values were determined by permutational multivariate analysis of variance (PERMANOVA). Statistical analyses were performed using R statistical software, R version 3.5.1, 2018 the R Foundation for Statistical Computing. A P<0.05 was considered to be statistically significant (adjusted for multiple comparison when performing multiple tests by False Discovery Rate (FDR)). All statistical tests were two-sided.

### Results

### Patient Characteristics and Laboratory Parameters

Shotgun sequencing of fecal samples from 81 patients with alcoholic hepatitis, 41 patients with alcohol use disorder, and 9 individuals who were not alcoholics (controls) (Table 4) was performed; and sequences aligned with those in the virulence factor gene database (VFDB)(42) (see Experimental Procedures). These patients with alcoholic hepatitis from 7 regions in Europe, the United States, and Mexico were enrolled in a multi-center observational study at 12 centers. The median hospitalization time before collection of fecal samples was 4 days (range, 0-24 days) (Table 5). The median model for end-stage liver disease (MELD) score of the patients with alcoholic hepatitis was 24; most patients with available liver biopsies had liver cirrhosis (Table 5).

Increased Carriage of Virulence-Related Genes in Fecal Samples from Patients with Alcoholic Hepatitis

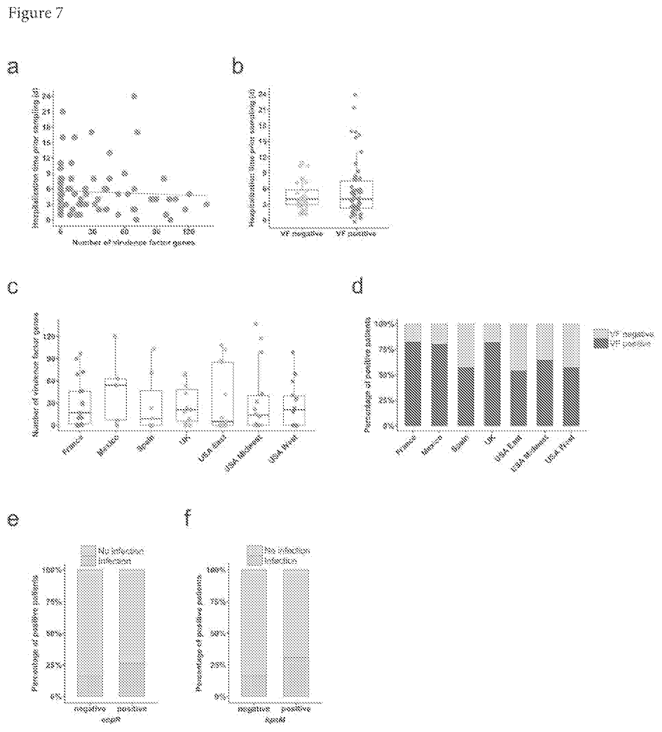

The core virulence factor genes included 350 different, experimentally verified genes that encode virulence factors (Chen et al., 2015). Significantly more virulence factors were found in fecal metagenomes from patients with alcoholic hepatitis than patients with alcohol use disorder or controls (FIG. 4A). The median hospitalization time of patients with alcoholic hepatitis was not associated with increased copies of genes encoding virulence factors, indicating that they were not acquired during hospitalization (FIGS. 7A and 7B). Overall, fecal samples from 68% of patients with alcoholic hepatitis had at least one virulence factor gene, compared with 29% of patients with alcohol use disorder and 11% of controls (P=2.75e-04 and 2.27e-03, respectively, by Fisher's exact test; FIG. 4B). There was heterogeneity in the host origin of virulence factor genes among the study subjects; the carriers usually carry several virulence factor genes originated from different host bacterial genera, independent from the center w here the patient was enrolled (FIG. 4C, FIGS. 7C and 7D). The most frequently observed virulence factors were derived from Escherichia, followed by Klebsiella, Shigella, Klebsiella/Yersinia, and then Enterococcus (FIGS. 4B and 4D).

Carriage of Virulence Factors Associate with Increased Mortality in Alcoholic Hepatitis Patients

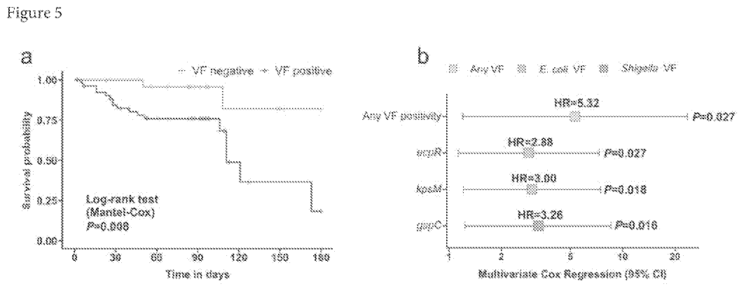

In patients with alcoholic hepatitis, intestinal carriage of virulence factor was associated with decreased probability of surviving 180 days (FIG. 5A); 82% of virulence factor-negative patients were alive after 180 days following enrollment, compared with 18% of virulence factor-positive patients (P=0.008). In the univariate Cox regression analysis, a fecal sample with any virulence factor gene, with any Escherichia virulence factor gene, or with 1 of 6 specific virulence factor genes was associated with 180-day mortality (Table 6). After being adjusted for antibiotic and steroid treatment in the multivariate Cox regression analysis, fecal samples with any virulence factor gene remained associated with increased risk of death, with an adjusted hazard ratio of 5.3 (95% CI 1.2-23.5; P=0.027; FIG. 2b). Specifically, among all 350 core virulence factor genes, fecal samples containing ecpR (E. coli), kpsM (E. coli), or gspC (Shigella dysenteriae) were independently associated with mortality (FIG. 5B).

Virulence Factors from E. coli Associate with Relative Abundance of E. coli

The strain-level phylogeny of ecpR- and kpsM-positive E. coli shows association with the commensal, rather than pathogenic lineages (FIGS. 6A and 6B; P=0.035 and 0.013, respectively, by Fisher's exact test); these virulence factor genes were not associated with infection immediately before or during hospitalization (FIGS. 7E and 7F. Table 5). Patients positive for ecpR and/or kpsM (32% of patients with alcoholic hepatitis) had a microbiome that differed significantly from that of non-carriers, when the relative abundance of fecal bacterial populations in the metagenomic dataset was analyzed (FIG. 6C). In particular, the presence of genes encoding virulence factors from E. coli is associated with the relative abundance of E. coli (FIG. 6D).

Relative E. coli Abundance and Virulence Factor Gene Carriage Each Contribute to the Risk of Death in Patients with Alcoholic Hepatitis

Higher E. coli abundance itself was independently associated with 180-day mortality; however, when adjusted for the detection of ecpR and/or kpsM, this association was no longer statistically significant (Table 7). This indicates that the abundance of E. coli and the carriage of genes encoding virulence factors each are associated with an increased risk of death in these patients.

Liver Cirrhosis and Intestinal Permeability are not Associated with Virulence Factor Carriage

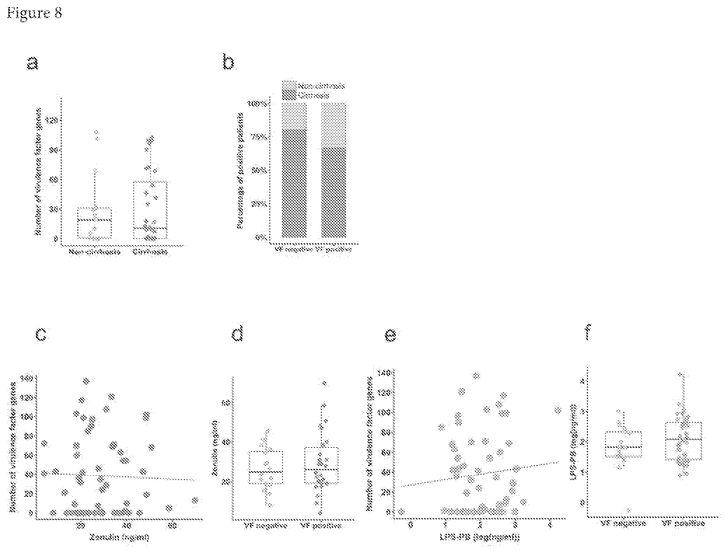

The presence of liver cirrhosis was not associated with higher numbers of virulence factor genes (FIGS. 8A and 8B). Furthermore, levels of zonulin (a marker for gut barrier dysfunction) and lipopolysaccharide binding protein (LPS-BP) (a marker of the systemic immune response to LPS) in serum samples were not associated with the number or presence of any virulence factor genes (FIGS. 8C-F). Virulence factors therefore do not seem to increase intestinal permeability or translocation of LPS.

### DISCUSSION

It was shown that the presence of virulence-related genes in commensal microbiota, and more specifically of two virulence factors encoded in the genome of Escherichia coli, are independently associated with mortality in patients with alcoholic hepatitis. This study may be the first to investigate the correlation between clinical outcomes and specific virulence-related genes in the gut metagenomes from patients with a metabolic and non-infectious disease. Among patients with liver cirrhosis, E. coli infections and in particular spontaneous bacterial peritonitis are frequent complications with high mortality (Arveniti et al., 2010). In this study, the presence of ecpR and kpsM in the gut microbiota was not associated with increased rates of infection. The broad phylogenetic distribution indicates that ecpR and kpsM presence is variable trait among E. coli isolates, and ecpR- or kpsM-positive E. coli are largely non-epidemic commensal strains. This is also consistent with the low frequency of ecpR and kpsM detection in fecal samples (22% and 31%, respectively), as epidemic isolates have typically higher frequencies of virulence genes (Bert et al., 2010).

Little is known about the specific environmental conditions leading to enrichment of virulence-related genes. Profound dysregulation of bile acid metabolism, which was observed in patients with alcoholic hepatitis (Brandl et al., 2018), could be one possible explanation for this expansion. The syndrome of alcoholic hepatitis develops rapidly and although most patients have underlying chronic liver disease, they are not repeatedly hospitalized. Liver failure was the main cause of death among the 18 patients who died within 180 days, followed by gastrointestinal bleeding; only 2 patients died from sepsis (Table 5). Therefore, mechanisms beyond infection contribute to the association between mortality and the presence of these virulence factors in patients with alcoholic hepatitis.

Metagenomic sequencing might not fully capture the presence of virulence factors among all bacterial populations in the gut; increased cytolysin in fecal samples from patients with alcoholic hepatitis, which was detected by quantitative PCR (Duan et al., 2019), was not observed by shotgun sequencing. Therefore, a combination of different techniques might be necessary to detect genes that encode virulence factors in bacteria of low abundance. The strength of the present study is that it included patients with alcoholic hepatitis participating in a multi-center observational trial, which reduced the chances that the virulence-related genes studied are region specific (FIG. 4C. FIGS. 7C and 7D).

Most studies investigating associations of the gut microbiota and disease have focused on diversity, relative abundance and metabolic profiles of specific microorganisms. Assessing the carriage of specific virulence factors adds an additional layer of complexity that might lead to new diagnostic biomarkers and treatments targets.

TABLE 4
Demographic and laboratory parameters of the study cohort

| | | | Alcohol use | | Alcoholic | |
|---|---|---|---|---|---|---|
| | Controls | | disorder | | hepatitis | |
| Variables | (n = 9) | | (n = 41) | | (n = 81) | |
| Sex (% male), n | 7 | (77.8) | 34 | (82.9) | 54 | (67.5) |
| (%), n = 130 | | | | | | |
| Age (years). | 51.0 | (27.0-71.0) | 44.0 | (27.0-67.0) | 51.3 | (30.0-75.0) |
| n = 130 | | | | | | |
| BMI (kg/m²), | 22.9 | (18.8-29.2) | 24.0 | (17.9-37.0) | 27.5 | (19.0-48.3) |
| n = 118 | | | | | | |
| Creatinine | | | 0.8 | (0.5-1.3) | 0.8 | (0.3-8.1) |
| (mg/dL), n = 118 | | | | | | |
| Bilirubin | | | 0.5 | (0.2-1.5) | 16.3 | (2.5-38.6) |
| (mg/dL), n = 118 | | | | | | |
| AST (IU/L), | | | 36.0 | (15.0-283.0) | 132.0 | (41.0-406.0) |
| n = 121 | | | | | | |
| ALT (IU/L), | | | 37.0 | (9.0-184.0) | 44.5 | (15.0-216.0) |
| n = 121 | | | | | | |
| Albumin (g/dL), | | | 4.5 | (2.2-5.2) | 2.4 | (1.3-4.1) |
| n = 112 | | | | | | |
| INR. n = 116 | | | 0.9 | (0.8-1.3) | 1.8 | (1.0-3.7) |
| GGT (IU/L), | | | 43.0 | (4.0-1131.0) | 188.0 | (33.0-3632.0) |
| n = 77 | | | | | | |
| Platelet count | | | 222.0 | (21.0-434.0) | 122.5 | (21.0-447.0) |
| (109/L), n = 122 | | | | | | |
| FIB-4, n = 115 | | | 1.3 | (0.4-21.4) | 8.0 | (1.4-66.3) |
| FIB-4 > 3.25 | | | 6 | (16.2) | 71 | (92.2) |

(F3-F4), n (%)

Notes:
values are presented as median with range in parentheses for continuous variables or number and percentage in parentheses for categorical variables. Percentages are calculated based on the actual number of patients in each group where the respective data was available. The number of subjects for which the respective data was available is indicated in the first column.

BMI, body mass index; AST, aspartate aminotransferase, ALT, alanine aminotransferase; INR, international normalized ratio; GGT, gamma-glutamyl transferase; MELD, model for end-stage liver disease; FIB-4, fibrosis-4 index

TABLE 5
Characteristics of patients with alcoholic hepatitis (n = 81)
Treatment at admission

| | | |
|---|---|---|
| Steroids, n (%), n = 79 | 31 | (39.2) |
| Pentoxifylline, n (%), n = 65 | 6 | (9.2) |
| Antibiotics, n (%), n = 79 | 40 | (50.6) |
| Proton pump inhibitors, n (%), n = 42 | 5 | (11.9) |

Clinical characteristics

| Hospitalization time prior sampling (d) | 4 | (0-24) |
|---|---|---|
| Infections, n (%), n = 68 | 13 | (19.1) |
| Blood stream infection | 7 | |
| Pneumonia | 4 | |
| Urinary tract | 3 | |
| Spontaneous bacterial peritonitis (SBP) | 2 | |
| Unknown | 1 | |
| Abstinent at day 30, n = 47 | | |
| Abstinent, n (%) | 40 | (85.1) |
| Reduced alcohol consumption, n (%) | 4 | (8.5) |
| Continued alcohol abuse, n (%) | 3 | (6.4) |

| | |
|---|---|
| Stage of Fibrosis, n (%), n = 45 0/1/2/3/4 | 2 (4.4)/0 (0.0)/5 (11.1)/ |
| | 6 (13.3)/32 (71.1) |

Clinical scores and outcome

| MELD, median (range), n = 79 | 23.7 | (11.7-45.9) |
|---|---|---|
| MELD > 21, n (%) | 62 | (78.5) |
| Child-Pugh stage, n (%), n = 78 A/B/C | 1 (1.3)/24 (30.8)/53 (67.9) | |
| Maddrey's DF, median (range), n = 65 | 59.3 | (−3.8-239.0) |
| Maddrey's DF > 32, n (%) | 57 | (87.7) |
| ABIC, median (range), n = 82 | 8.3 | (5.5-11.0) |
| 30 day mortality rate, n (%), n = 73 | 8 | (11.0) |
| 90 day mortality rate, n (%), n = 53 | 13 | (24.5) |

Causes of death within 180 days

| | |
|---|---|
| Liver failure* | 10 |
| Gastrointestinal bleeding | 3 |
| Septic shock | 2 |
| Unknown | 3 |

*One patient underwent liver transplantation.
Notes:
antibiotics include prophylactic antibiotics. Values are presented as median (range) for continuous variables or number (percentage) for categorical variables. Percentages are calculated based on the actual number of patients in each group where the respective data was available. Individual persons can have more than reason for infections. The number of subjects for which the respective data was available is indicated in the first column.

TABLE 6
Univariate Cox regression analysis of virulence factors associated
with 180-day mortality in patients with alcoholic hepatitis reason
for infections. The number of subjects for which the respective
data was available is indicated in the first column.

| Univariate Cox regression | Organism | HR | 95% CI | P value* |
|---|---|---|---|---|
| Any virulence factor | | 5.9 | 1.3-25.7 | 0.019 |
| positivity | | | | |
| Escherichia virulence | | 2.9 | 1.0-8.1 | 0.041 |
| factor positivity | | | | |
| ecpR. | E. coli | 3.0 | 1.2-7.5 | 0.019 |
| ecpD | E. coli | 2.7 | 1.1-6.8 | 0.031 |
| kpsM | E. coli | 3.0 | 1.2-7.5 | 0.017 |
| ybtX | K. pneumoniae/ | 2.7 | 1.1-6.7 | 0.033 |
| | Y. pestis | | | |
| gspC | S. dysenteriae | 3.5 | 1.4-9.0 | 0.010 |
| chut | E. coli/S. | 2.7 | 1.1-6.7 | 0.034 |
| | dysenteriae | | | |

*Significance is indicated in bold.
Notes:
patients were censored at the time point they were last seen alive. P values were determined by two-sided Wald test.
HR, hazard ratio;
CI, confidence interval

TABLE 7
Multivariate Cox regression analysis of virulence factors associated

| Multivariate Cox | | | |
|---|---|---|---|
| regression | HR | 95% CI | P value* |
| E. coli abundance | 1.06 | 1.02-1.11 | 0.010 |
| (percent increase) | | | |
| E. coli abundance | 1.04 | 0.99-1.10 | 0.106 |
| (adjusted for VFs) | | | |

*Significance is indicated in bold.
Notes:
Adjusted for treatment with antibiotics, steroids, and the sequence batch (2 different runs). In the second row, E. coli abundance was additionally adjusted for the presence of the VFs ecpR and/or kpsM. Patients were censored at the time point they were last seen alive. P values were determined by two-sided Wald test.
HR, hazard ratio;
CI, confidence interval

### Example 3

In one embodiment, the ecpR gene that is detected encodes

(SEQ ID NO: 1)
MTWQSDYSRDYEVKNHMECQNRSDKYIWSPHDAYFYKGLSELIVD
IDRLIYLSLERIRKDEVFINESTDSLSEFINRDNEWLSAVKGRQV
VLIAARKSBALANYWYYNSNIRGVVYAGLSRDIRKELVYVINGRE
LRKDIKKDKITDREMEIIRMTAQGMQPKSIARIENCSVKTVYTAR
RNARAKLYSKIYKEVQ

or a polypeptide having at least 80%, 85%, 92%, 94%, 95%, 97%, 98%, or 9% amino acid sequence identity thereto.

In one embodiment, the methods detect nucleic acid specific for at least a portion of

(SEQ ID NO: 2)
gtgacatggc aaagtgatta cagtagggac tatgaggtta
aaaaccatat ggaatgtcaa aacccttctg ataaatacat
ctggtctccc catgacgcct acttctataa aggactatct
gaactgattg tggatatcga cagattaatt tatctatcgt
tcgagaaaat tagaaaagat ttcgtgttta tcaatctcag
tacggattct ttatctgaat ttataaaccg tgataatgaa
tggttatccg cggtaaaggg gaaacaggtc gtattgattg
cggccagaaa gtcagaggcc ttagcaaatt attggtatta
caatagcaat attaggggcg tggtatacgc tggactgagt
cgtgatatta gaaaagaact ggtctatgtg attaatggca
ggttcctgag aaaagatatt aagaaagata aaatcacgga
ccgggaaatg gaaattatcc gcatgacggc ccagggaatg
caacctaaat cgattgccag aattgaaaat tgtagtgtga
agacagtgta tacccatcgg cgtaatgctg aggccaagct
gtactcaaaa atatataagt tggttcagta a

or the complement thereof, or a polynucleotide having at least 80%, 85%, 90%, 92%, 94%, 95%, 97%, 98%, or 99% nucleic acid sequence identity thereto.

In one embodiment, the gene that is detected encodes KpsM having

(SEQ ID NO: 3)
mtwqsdysrd yevinhmecq nesdkyiwsp
hdayfykgls elivdidrli ylslekirkd
fvfinistds lsefinidne wisavkgkqv
vliaarksea lanywyynsn irgyvyagls
rdickelvyv ingrflekdi kkdkitdrem
eiirmtaqgm qpksiacien esvktvythe
rnaeaklysk iyklvq

or a polypeptide having at least 80%, 85%, 90%, 92%, 94%, 95%, 97%, 98%, or 99% amino acid sequence identity thereto.

In one embodiment, the gene that is detected encodes KpsM having

(SEQ ID NO: 4)
MARSGFEVQKVTVEALFLREIRTRFGKFRLGYLWAILEPSAHLLI
LLGILGYVMHRTMPDISFPVFLLNGLIPFFIFSSISKRSIGAIEA
NQGLFNYRPVKPIDTIIARALLETLIYVAVYILLMLIVWMTGEYF
EITNFLQLVLTWSLLIILSCGVGLIFMVVGKTFPEMQKVLPILL
KPLYFISCIMFPLHSIPKQYWSYLLWNPLVHVVELSREAVMPGY
ISEGVSLNYLAMFTLVTLFIGLALYRTREEAMLTS

or a polypeptide having at least 80%, 85%, 90%, 92%, 94%, 95%, 97%, 98%, or 99% amino acid sequence identity thereto.

In one embodiment, the methods detect nucleic acid specific for at least a portion of

(SEQ ID NO: 5)
gattaggatc acatcatcaa atggcaagaa gtggatttga
agttcagaaa gtcaccgtag aggcattatt tctacgagaa
atacgaacac gctttggtaa gtttcgtctg gggtatttgt
gggcgattct tgaaccctcc gcgcatttgc tgatactgtt
gggaattttg ggttacgtta tgcaccgcac tatgccagac
atctcgttcc cggtgttttt acttaatggc ctgattccct
tttttatctt tagtagtatt agcaaacgtt ctattggtgc
tattgaagcg aaccagggac tgtttaatta tcgaccagta
aaacccatcg atacgatcat tgcacgtgca ctgcttgaga
cactgattta cgttgctgtt tatattttgc tcatgcttat
cgtctggatg acaggcgaat atttcgaaat tacaaacttt
ttacaacttg tgctcacctg gagtttgtta atcattcttt
catgtggcgt cggcttaata tttatggtcg ttggtaaaac
ctttcctgaa atgcaaaagg tcctgccgat actgcttaag
ccgctgtatt tcatctcctg catcatgttc cctctacact
cgattccaaa acaatactgg tcatatctac tctggaaccc
attagtgcat gttgtggagt taagccgcga ggcagttatg
cctggctata tcagtgaagg cgtgagtctg aactaccttg
caatgtttac tctggtcacc ctgttcatcg gcctggcatt
ataccgaacg cgtgaagagg caatgctgac atcatga

or the complement thereof, or a polynucleotide having at least 80%, 85%, 90%, 92%, 94%, 95%, 97%, 98%, or 99% nucleic acid sequence identity thereto.

### REFERENCES

Arvaniti et al., Gastroenterology, 139:1246 (2010).
Ball et al., J. Abn. Psych., 106:545 (1997).
Bert et al., J. Clin. Microb., 48:709 (2010).
Blackburn et al., J. Clin Microb., 47:1781 (2009).
Brandl et al., J. Hepatology, 69:396 (2018).
Burrack & Higgins, Curr. Opin Microb., 10:4 (2007).
Caspi et al., Nucleic Acids Res., 46:D633 (2018).
Caussy et al., Hepatology, 68:918 (2018).
Chen et al., Hepatology, 54:562 (2011).
Chen et al., Hepatology, 61:883 (2015).
Chen et al., Nucleic Acids Res., 44:D694 (2015).
Chen et al., Nucleic Acids Res., 44:D694 (2016).
Chu et al., J. Hepatology, ______:______ (2019).
Cirioni et al., Antimir. Agents Chemo., 51:4518 (2007).
Dominguez et al., Am. J. Gastroenterology, 103:2747 (2008).
Duan et al. (Nature, 575:505 (2019).
Forrest et al., Gut., 54:1174 (2005).
Forster et al., Nature Biol., 37:186 (2019).
Franzosa et al., Nat. Methods, 15:962 (2018).
Gao et al., Dig. Dis Sci., 64:1878 (2019).
Hochhut et al., Contri. Microb., 12:234 (2005).
Hunt et al., Microbial Genomics, 3:e000131 (2017).
Kim et al., J. Clin. Invest., 90:897 (1992).
Lang et al., Hepatology, ______:______ (2019).
Lee & Schneewind, Genes & Dev., 15:1725 (2001).
Lee et al., JAMA Internal Med., 179:340 (2019).
Llopis et al., Gut., 65:830 (2016).
Lozano et al., The Lancet, 380:2095 (2012).
Lucey et al., N. Engl. J. Med., 360:2758 (2009).
Maddrey et al., Gastroenterology, 75:193 (1978).
Mathurin et al., Hepatology, 38:1363 (2003).
Mathurin et al., J. Hepatology, 56:S39 (2012).
Parks et al., Nature Biot., 36:996 (2(18).
Philips et al., Clin. Gastr. Hepatology, 15:600 (2017).
Ponziani et al., Hepatology, 69:107 (2019).
Puri et al., Hepatology, 67:1284 (2018).
Rehm et al., Alcoholism: Clinical and Experimental Research, 38:1068 (2014).
Rehm et al. J. Hepatology, 59:160 (2013).
Sarin et al., J. Hepatol., 70:260 (2019).
Seitz et al., Nat. Rev. Dis. Primers, 4:16 (2018).
Seki & Schwabe, Hepatology, 61:1066 (2015).
Shao et al., Nature, 574:117 (2019).
Stamatakis, Bioinformatics, 30:312 (2014).
Stärkel & Schnabl, Semin. Liver Dis., 36:331 (2016).
Thursz et al., N. Engl. J. Med., 372:1619 (2015).
Truong et al., Genome Res., 27:626 (2017).
Truong et al., Nat. Methods, 12:902 (2015)
Uesugi et al., Hepatology, 34:101 (2001).
Wood & Salzberg, Genome Biol., 15:R46 (2014).
Zhong et al., Hepatology, ______:______ (2019).

All publications, patents and patent applications are incorporated herein by reference while in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method of diagnosing a patient in need of treatment or diagnosing alcoholic hepatitis or other chronic liver disease in a patient, comprising detecting in a sample of the patient the presence or amount of E. coli expressing ecpR and/or kpsM or detecting in a sample of the patient the presence or amount of ecpR and/or kpsM.

2. The method of claim 1 wherein the patient has hepatitis or other chronic liver disease.

3. The method of claim 1 wherein the presence or amount of ecpR and/or kpsM nucleic acid in the sample is detected.

4. The method of claim 3 wherein the presence or amount is detected by sequencing or by a nucleic acid amplification reaction.

5. (canceled)

6. The method of claim 1 wherein the presence or amount is detected in a fecal sample.

7. (canceled)

8. The method of claim 1 wherein EcpR and/or KpsM protein is detected.

9. The method of claim 1 wherein phage is employed to detect the E. coli expressing ecpR and/or kpsM.

10. The method of claim 9 wherein the phage is not a lytic phage.

11. The method of claim 1 wherein the presence or the amount the E. coli expressing ecpR and/or kpsM, or the presence or the amount of ecpR and/or kpsM in the sample relative to a control that lacks E. coli expressing ecpR and/or kpsM genes or that lacks ecpR and/or kpsM genes, is indicative of alcoholic hepatitis or other chronic liver disease in the patient.

12-14. (canceled)

15. A method to treat a mammal having hepatitis or other chronic liver disease, comprising: administering to a mammal having hepatitis or other chronic liver disease a composition comprising an effective amount of bacteriophage selected to target E. coli expressing ecpR and/or kpsM.

16. The method of claim 15 wherein the mammal is a human.

17. The method of claim 15 further comprising monitoring levels of E. coli expressing ecpR and/or kpsM or levels of ecpR and/or kpsM after administration of the composition.

18. The method of claim 15 wherein a cocktail of lytic phage is administered.

19. The method of claim 15 wherein the composition is orally administered.

20-21. (canceled)

22. The method of claim 15 wherein the mammal is a human having alcoholic hepatitis.

23. (canceled)

24. The method of claim 22 wherein a cocktail of lytic phage is administered.

25. The method of claim 22 wherein the composition is orally administered.

26-27. (canceled)

28. The method of claim 15 further comprising: detecting over time the presence or an amount of E. coli expressing ecpR and/or kpsM, or the presence or amount of expression of ecpR and/or kpsM, in a fecal, liver or blood sample of the mammal; and determining whether the amount over time increases, thereby detecting progression of liver disease or monitor liver disease therapy in a mammal.

29-35. (canceled)

36. A method comprising:
detecting the presence or amount of E. coli expressing ecpR and/or kpsM in a fecal sample of a human; and
treating a human having E. coli expressing ecpR and/or kpsM or an increased amount of E. coli expressing ecpR and/or kpsM relative to a control sample, with an anti-hepatitis therapy or a liver disease resolving therapy.

37. The method of claim 36 wherein the therapy is corticosteroid administration, pentoxifylline administration or a liver transplant.
